(19) United States
(12) Patent Application Publication
(10) Pub. No.: US 2011/0224574 A1
(43) Pub. Date: Sep. 15, 2011

(54) METHODS AND SYSTEMS FOR TISSUE PROCESSING AND IMAGING

(76) Inventors: John W. Sadler, Palo Alto, CA (US); Jonathan W. Mulholland, Palo Alto, CA (US); Tadmor Shalon, Palo Alto, CA (US)

(21) Appl. No.: 13/063,948

(22) PCT Filed: Nov. 13, 2009
(86) PCT No.: PCT/US2009/064315

§ 371 Date: May 31, 2011

Related U.S. Application Data
(60) Provisional application No. 61/114,305, filed on Nov. 13, 2008.

(51) Int. Cl. A61B 10/00; C12Q 1/02; C12M 1/34

(52) U.S. Cl. 600/562; 435/29; 435/288.7

(57) ABSTRACT

The disclosure relates generally to methods and systems for processing biological samples obtained from a subject having, or believed to have, a cell proliferation disorder. The disclosure further relates to methods and systems for detecting an object of interest in a biological sample.

### CROSS REFERENCE TO RELATED APPLICATIONS

This application claims priority to U.S. Provisional Application No. 61/114,305, titled “Methods and Systems for Tissue Processing and Imaging,” filed Nov. 13, 2008. This application is herein incorporated by reference in its entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### TECHNICAL FIELD

The disclosure relates generally to methods and systems for processing biological samples obtained from a subject having, or believed to have, a cell proliferation disorder. The disclosure further relates to methods and systems for detecting an object of interest in a biological sample.

### BACKGROUND OF THE INVENTION

In the field of medical diagnostics and research including oncology, the detection, identification, quantification, and characterization of cells of interest, such as cancer cells, through testing of biological samples is an important aspect of diagnosis and research. Typically, a biological sample such as tissue obtained from skin, lymph nodes, fine needle aspirates, or other biological materials are processed by staining a sample and detecting objects of interest, such as cells of interest, associated with the processed sample.

### SUMMARY OF THE INVENTION

The disclosure relates to methods and systems for the rapid processing of a tissue sample obtained from a surgical procedure. The processing is performed in a manner that provides for the identification of objects of interest within a tissue sample so that critical information regarding disease detection is obtained and transferred to a health care provider. In some implementations, a method and system can detect, through the use of specific tissue staining reagents and microscopy techniques, the presence and morphology of neoplastic cells associated with a tissue sample.

In a first embodiment, a method for processing and imaging a tissue sample is provided. The method includes optionally capturing an image of a surgical site associated with a subject. In some aspects, the surgical site includes at least one identifier suitable for orienting a tissue sample associated with the surgical site. The method further includes excising the tissue sample and immobilizing the sample in a carrier suitable for manipulating a target surface associated with a field of view. Prior to, concomitant with, or subsequent to immobilization, the sample is contacted with one or more reagents suitable for facilitating the imaging of an object of interest by microscopy. In some aspects the microscopy is fluorescence microscopy. In other aspects, the tissue sample is not frozen or otherwise modified by a pliability-reducing treatment. In another aspect, the tissue sample is not sectioned prior to contacting with the one or more reagents. In yet another aspect, the one or more reagents penetrate the target surface of the sample to a predetermined depth. The method further includes irradiating the target surface at one or more wavelengths selected to excite the one or more reagents associated with the target surface and acquiring images of one or more regions of the target surface to identify an object of interest associated with the target surface. The method further includes determining the position of the object of interest relative to the surgical site, displaying the object of interest on a display, and optionally storing any imaging information obtained during the process.

In a second embodiment, images obtained from a method of the first embodiment are transmitted to a remote user that verifies the presence of an object of interest in any of the images. The remote user further identifies the image, or plurality of images, that best depicts the position of the object in the tissue sample and correlates the identification with the identifier to determine the position of the object relative to the surgical site. The remote user optionally annotates the image or plurality of images identified by delineating the detected object of interest. An annotated image that includes at least one demarcation alerting a health care provider to the position of the detected object is transmitted to a health care provider.

In a third embodiment, a method for substantially reducing the amount of time needed to prepare and image an excised tissue sample is provided. The method includes preparing and imaging the sample by immobilizing the tissue sample in a carrier suitable for manipulating a target surface associated with a field of view and contacting the sample with one or more reagents suitable for facilitating the imaging of an object of interest by microscopy. In some aspects, the microscopy is fluorescence microscopy. In other aspects, the sample is not sectioned or substantially modified by a pliability-reducing treatment. The method further includes irradiating the target surface at one or more wavelengths selected to excite the one or more reagents associated with the target surface and acquiring images of one or more regions of the target surface. The method further includes identifying an object of interest associated with the target surface, displaying the object of interest on a display, and optionally storing the acquired images.

In a fourth embodiment, a system for processing and imaging a tissue sample is provided. In general, a system includes a carrier suitable for manipulating a target surface associated with a tissue sample obtained from a surgical site and a radiation source configured to direct input radiation, at one or more wavelengths, to a target surface associated with the tissue sample. A detector assembly configured to capture radiation generated by the reagents associated with the target surface is also included. Optionally, the system includes a controller operably associated with the detector assembly. In some implementations the controller is configured to acquire a plurality of images of one or more regions of the target surface. A system also includes a display element associated with the detector assembly and controller. The display element allows a user to visualize an object of interest and correlate the position of the object with the identifier to determine the position of the object of interest relative to a surgical site. In other implementations, a system optionally includes a storage element for storing imaging information obtained from the detector assembly.

### BRIEF DESCRIPTION OF THE DRAWINGS

The above and other features of the disclosure including various details of construction and combinations of parts will now be more particularly described with reference to the accompanying drawings and pointed out in the claims. It will be understood that the particular method and system embodying the disclosure is shown by way of illustration only and not as a limitation of the disclosure. The principles and features of this disclosure may be employed in varied and numerous embodiments without departing from the scope of the disclosure.

FIG. 1 illustrates an exemplary system 100 in which embodiments may be implemented.

FIG. 2 illustrates a comparative operational flow 200 and an exemplary operational flow 300 related to methods and systems for analysis of a tissue sample.

FIG. 3A illustrates an emission profile for an exemplary reagent.

FIG. 3B illustrates a logarithmic emission profile for an exemplary reagent.

FIG. 4 illustrates an emission profile for an exemplary reagent at various excitation wavelengths.

FIG. 5A illustrates normal tissue associated with a tissue sample imaged by methods and systems described herein.

FIG. 5B illustrates neoplastic tissue associated with a tissue sample imaged by methods and systems described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Provided herein are methods and systems applicable for the identification of objects of interest, such as neoplastic cells, associated with a tissue sample obtained from a subject having, or believed to have, a cell proliferation disorder. In general, the methods and systems provide rapid and accurate screening of tissue samples for the identification of cellular morphology and structure indicative of cell proliferative disorder. In some implementations a tissue sample is processed in a manner that does not require sectioning and/or pliability-reducing treatment in order to facilitate examination of a target surface. In other implementations, a tissue sample is treated with reagents that facilitate the identification of objects of interest by microscopy, such as confocal microscopy. The methods and systems are useful for identifying objects of interest in the margins of tissue samples obtained from, e.g., skin, breast or lymph tissue. With regard to a skin sample, the methods and systems are suitable for providing rapid processing of tissue and enhanced images of objects of interest such as cells, or cell clusters, associated with basal cell carcinoma (BCC) or squamous cell carcinoma (SCC). In some implementations the images are generated from a tissue sample excised from a surgical site by e.g., needle biopsy or Mohs micrographic surgery.

Provided herein are methods for processing and imaging a tissue sample. In one implementation, a method includes optionally capturing an image of a surgical site associated with a subject. In some aspects, the surgical site includes at least one identifier suitable for orienting a tissue sample associated with the surgical site. The method further includes excising the tissue sample and immobilizing the sample in a carrier suitable for manipulating a target surface associated with a field of view. Prior to, concomitant with, or subsequent to immobilization, the sample is contacted with one or more reagents suitable for facilitating the imaging of an object of interest by microscopy. In some aspects the microscopy is fluorescence microscopy. In other aspects, the tissue sample is not frozen or otherwise modified by a pliability-reducing treatment. In another aspect, the tissue sample is not sectioned prior to contacting with the one or more reagents. In yet another aspect, the one or more reagents penetrate the target surface of the sample to a predetermined depth. The method further includes irradiating the target surface at one or more wavelengths selected to excite the one or more reagents associated with the target surface and acquiring images of one or more regions of the target surface to identify an object of interest associated with the target surface. The method further includes determining the position of the object of interest relative to the surgical site, displaying the object of interest on a display, and optionally storing any imaging information obtained during the process.

The methods provided herein decrease the amount of time needed to process and image a tissue sample by avoiding the need to section the sample prior to staining and imaging. The methods utilize a unique combination of sample processing and imaging techniques to examine the margins of a tissue sample and identify an object of interest. In some implementations, confocal laser fluorescent microscopy may be used to generate 3-dimensional stereoscopic image information of a target surface treated with reagents suitable for fluorescence imaging. Images may be obtained at a minimum resolution. Exemplary image resolutions of about 0.5 μm to about 3 μm are achievable using the present methods and systems. The methods and systems provided herein enable a health care professional to quickly and efficiently assess the state of a sample derived from a patient, thereby reducing the amount of time a patient is required to wait for a diagnosis. Such a reduction in time translates into a reduction in costs associated with maintaining a patient in a procedure-ready environment should additional tissue excision be required. The reduction in time also translates into a reduction in patient anxiety because the diagnosis is rendered more quickly and any additional treatments may be initiated immediately.

Example methods may be better appreciated with reference to flow diagrams. While for purposes of simplicity of explanation, the illustrated methods are shown and described as a series of blocks, it is to be appreciated that the methods are not limited by the order of the blocks, as some blocks can occur in different orders and/or concurrently with other blocks from that shown and described. Moreover, less than all the illustrated blocks may be required to implement an example method. In some examples, blocks may be combined, separated into multiple components, may employ additional, not illustrated blocks, and so on. In some examples, blocks may be implemented in logic. In other examples, processing blocks may represent functions and/or actions performed by functionally equivalent circuits (e.g., an analog circuit, a digital signal processor circuit, an application specific integrated circuit (ASIC)), or other logic device. Blocks may represent executable instructions that cause a computer, processor, and/or logic device to respond, to perform an action(s), to change states, and/or to make decisions. While the figures illustrate various actions occurring in serial, it is to be appreciated that in some examples various actions could occur concurrently, substantially in parallel, and/or at substantially different points in time.

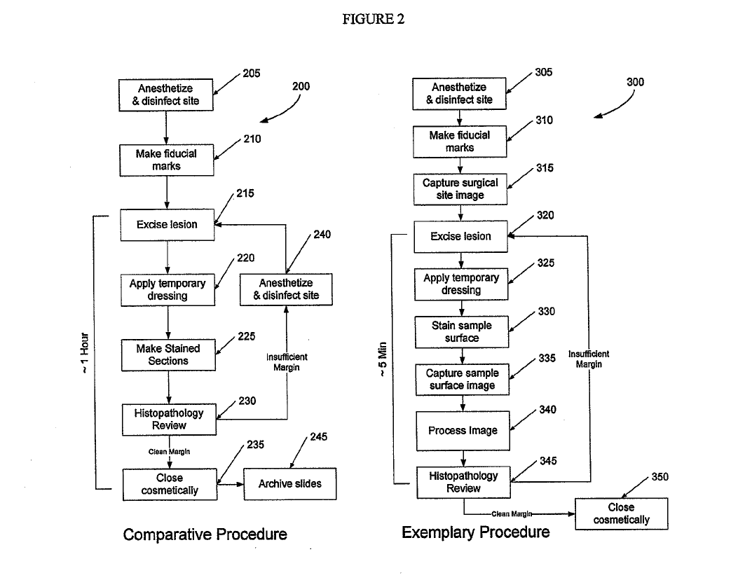

FIG. 2 illustrates a flow diagram of a comparative procedure 200 and a flow diagram of an exemplary procedure 300. Comparative procedure 200 provides an overview of currently available methods of excising, treating and imaging a tissue sample obtained from a surgical site. In general, the method includes anesthetizing and disinfecting a surgical site 205 and adding an identifier, such as a fiducial mark 210, to the site. A tissue sample, such as a lesion, is excised 215 by a health care professional and a temporary dressing is applied to the surgical site 220. The tissue sample is sectioned and stained 225. The stained sections then undergo histopathology review 230 to identify the presence or absence of an object of interest, such as a neoplastic cell or group of neoplastic cells. If the margins of the sample are free of an object of interest then the surgical site is closed 235 and images of the stained section(s) are archived for future review 245. However, if an object of interest is detected, then the procedure is repeated 240 until the margins are clean, e.g., free of objects of interest. The previously described method generally requires an hour or more to complete the excision 215, staining 225 and review 230 steps.

Identifying the extent to which cancer cells have migrated from a primary site to secondary sites is critical to surgical procedures charged with removal of all neoplastic cells from a surgical site. Mohs micrographic surgery is an example of a surgical procedure that involves the precise excision of a cancer with minimal damage to the surrounding normal skin. Conventionally, precise excision is guided by histopathologic examination for cancer margins in the excised tissue slices during Mohs surgery. Typically, 2-4 slices are excised, and there is a waiting time of 45 minutes or longer for the surgeon and patient while each slice is processed and imaged by conventional microscopy. Other surgical procedures for obtaining a tissue sample for biopsy, such as a needle biopsy of breast tissue, require even longer periods of time to process and image margins of a tissue sample.

In contrast, and referring again to FIG. 2, exemplary procedure 300 describes a method which dramatically reduces the amount of time needed to process a tissue sample obtained from a surgical site. A surgical site is anesthetized and disinfected 305. An identifier, such as a fiducial mark 310, is optionally associated with the site. An image of the surgical site is optionally captured 315. A tissue sample, such as a lesion, is excised 320 by a health care professional and a temporary dressing is applied to the surgical site 325. The tissue sample is stained or otherwise contacted with a reagent that facilitates imaging of a target surface associated with the sample 330. Images of the target (sample) surface are captured 335 and the images are optionally processed 340. The images undergo histopathology review 345 to identify the presence or absence of an object of interest, such as a neoplastic cell or group of neoplastic cells. If the margins of the sample are free of an object of interest then the surgical site is closed 350. However, if an object of interest is identified, then the procedure is repeated until the margins are clean, e.g., free of objects of interest. Various implementations of exemplary procedure 300 may require 3, 4, 5, 6, 7, 8, 10, 12 or 20 minutes to complete the excision 320, staining 330, imaging 335 and review 345 steps. As described in the exemplary embodiments, tissue processing time (e.g., immobilizing the tissue sample in a carrier and contacting the sample with one or more reagents suitable for facilitating the imaging of the target surface) and image acquisition time (e.g., acquiring images the target surface and identifying an object of interest associated with the target surface) are functions of the area of the sample surface under examination. Sample processing and image acquisition time may be less than about 50, 40, 30, 20 or 10 seconds per cm² of imaged target surface. It is understood that the acquisition time may depend upon the resolution of the area under examination and the scanning rate of the detector assembly. In some implementations, the total time from sample immobilization to the initiation of image acquisition is less than about 50, 40, 30, 20, or 10 seconds per cm² of imaged target surface. Accordingly, the methods and systems provided herein represent a substantial decrease in the amount of time needed to examine a tissue sample for objects of interest.

The term “reagent,” as used herein includes any compound or composition that facilitates the detection of an object of interest during examination of a tissue sample. In one implementation, a reagent facilitates the detection of an object of interest undergoing microscopy. In another implementation, a reagent facilitates the detection of an object of interest undergoing confocal laser microscopy. For example, reagents used to facilitate imaging of a tissue sample obtained from Moh's surgery or a lumpectomy may be chosen to mimic the traditional hematoxylin and eosin (H&E) staining technique. This may be accomplished using fluorescent reagents that stain “acidic” components of a cell such as the nucleus, and “basic” components of a cell, such as the cytoplasm.

Accordingly, the term “reagent” encompasses acid stains capable of staining basic structures. An exemplary acid stain includes Eosin which is both a chromophore and a fluorophore and may be used to facilitate visualization of the cytoplasm. In general, the excitation of Eosin may occur at a radiation wavelength between about 480 nm and about 600 nm, between about 500 nm and about 580 nm, or about 514 nm and about 545 nm.

Additional reagents include those that stain nucleic acids. Such reagents include DRAQ5 which may be used as a nuclear stain because of its affinity for nucleic acids. Additional nucleic acid stains are known to the skilled artisan. In general, the excitation of DRAQ5 may occur at a radiation wavelength between about 560 nm and about 680 nm, between about 600 nm and about 660 nm, or about 633 nm and about 650 nm. The excitation of DRAQ5 may occur at about 647 nm. Additional excitation wavelengths for DRAQ5 include 488 nm, 514 nm, 568 nm and 633 nm. The emission profile of DRAQ includes >665 nm to infra-red>800 nm. The Emλₘₐₓ for DRAQ5 intercalated with a double-stranded (ds) nucleic acid, such as dsDNA, is about 681 nm to about 697 nm. In general, the emission profile has minimal overlap with visible range reagents such as e.g. green fluorescent protein (GFP) and fluorescein isothiocyanates FITC). A system provided herein may include emission filters such as one or more low pass filter(s) at 695LP, 715LP or 780 LP. DRAQ5 is compatible with optics of bench-top flow, laser scanning cytometers and non-UV laser scanning and lamp-based confocal microscopes. Additionally, DRAQ5 has a high quantum efficiency (QE), requires no washing or limited amounts of washing, and is photostable.

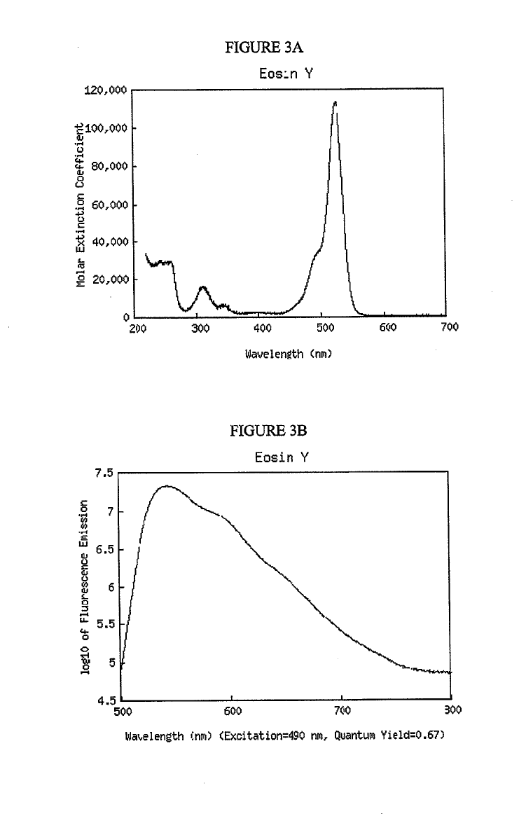

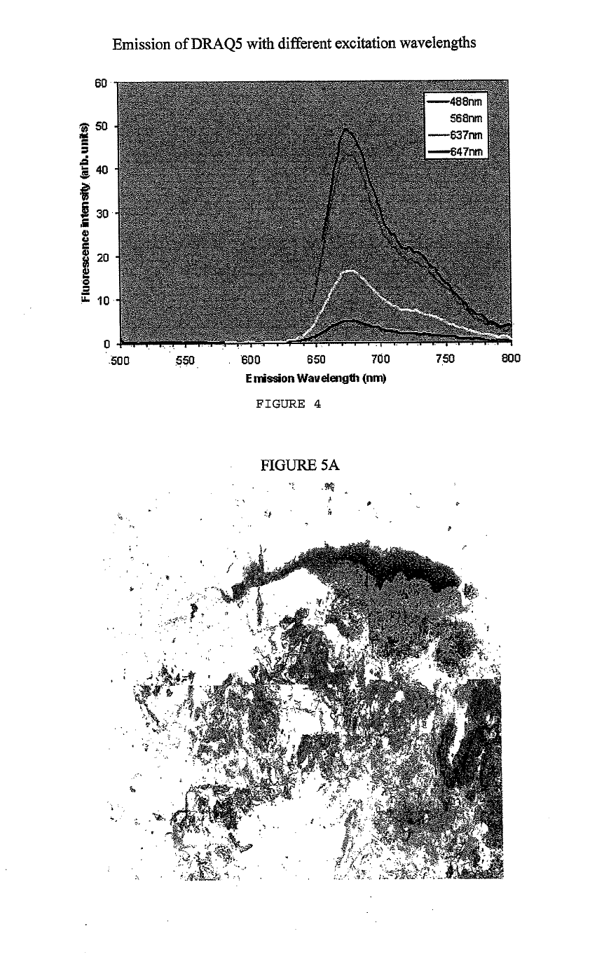

In implementations where more than one fluorescent reagent is used to image a tissue sample, it is desirable to use reagents that have different emission spectra so that they may be detected as two separate signals. Both signals may be detected individually or simultaneously during multiple or single image acquisition scans of a tissue sample. For simultaneous detection, a detector assembly may include a dual detection system. In some implementations, multiple reagents may be excitable by the same radiation source, such as a laser. Accordingly, multiple reagents may have overlapping excitation spectra, but separated emission spectra. Emission spectra associated with the exemplary reagents discussed above are provided in FIGS. 3A, 3B, and 4. Additional reagents useful in the present methods include orange or red dyes series SYTO® as well as LDS 751® available from Invitrogen®. Additional reagents known to the skilled artisan may be used as a stain in various implementations described herein. Accordingly, a reagent useful in the present methods includes, but is not limited to, the reagents specifically identified in this document.

In other implementations, a reagent includes those compounds that specifically bind to a marker, such as a cellular molecule, present in a tissue sample. A marker can be any cell component present in a sample that is identifiable by known microscopic, histologic, or molecular biology techniques. Markers can be used, for example, to distinguish neoplastic tissue from non-neoplastic tissue and/or one cell type from another cell type. Such markers can also be used to identify a molecular basis of a disease or disorder including a neoplastic disease or disorder. Such a marker can be, for example, a molecule present on a cell surface, an over-expressed target protein, a nucleic acid mutation or a morphological characteristic of a cell present in a sample.

In some implementations, a tissue sample may be contacted with primary and secondary labels to detect objects of interest. Useful primary and secondary labels include spectral labels such as green fluorescent protein, fluorescent dyes (e.g., fluorescein and derivatives such as fluorescein isothiocyanate (FITC) and Oregon Green™, rhodamine and derivatives (e.g., Texas red, tetrarhodamine isothiocyanate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P, etc.), enzymes (e.g., horseradish peroxidase, alkaline phosphatase etc.), spectral calorimetric labels such as colloidal gold or colored glass or plastic (e.g. polystyrene, polypropylene, latex, etc.) beads. The label can be coupled directly or indirectly to a detection reagent, such as an antibody or antigen, according to methods well known in the art. As indicated above, a wide variety of labels may be used, with the choice of label depending on sensitivity required, ease of conjugation with the compound, stability requirements, available instrumentation, and disposal provisions.

Reagents may be used to stain a tissue sample excised from a surgical site. Contacting a tissue sample with a reagent may be accomplished by any process known to the skilled artisan. Exemplary processes include spraying the reagent on the tissue sample or submerging the sample in a bath that includes the reagent. Alternatively, a reagent may be rolled onto e.g., a porous or hydrophilic platen. The tissue may be pressed against the platen to transfer controlled quantities of the reagent. In some implementations, a vacuum may be applied to the tissue sample before or during application of the reagent so that reagent penetration may be controlled or enhanced. In some cases the use of a dense gas or supercritical fluid may facilitate transport of reagent into a tissue mass. In yet other cases the use of a solvent other than water may be preferred for the transport of the reagent. Mechanisms by which the dye molecule transport can be facilitated include the application of an external electric, magnetic, sonic or other field to promote molecule transport across tissue and speed of transport of dye molecules, for example the use of an iontophoretic mechanism. Additional processes for applying a reagent include controlling the temperature of the tissue sample and reagent. The amount, type and temperature of the reagent may be varied with the density of the tissue sample. The density of the tissue sample may be based on empirical calibration. In addition, the staining protocol may be based on observed optical density change of the tissue after a short calibration bath in one or each reagent. An initial examination of the tissue sample during or after contact with a reagent may be used to determine if the contacting process has been completed appropriately. Alternatively, a staining process may be iterated until a desired depth of penetration or optical density (OD) is reached.

To control the depth of penetration of a dye into a tissue sample various strategies may be employed. One method for controlling depth of penetration is to covalently link the dye molecule to the tissue receptor site through means of a catalyzed chemical reaction sometime after the introduction of the dye into the tissue. In one example the dye molecule is derivatized with a photoactivated chemical moiety capable of producing a free radical upon exposure to UV light. Many examples are known to those skilled in the art. The derivatized molecule may be allowed to passively diffusive into the tissue for a predetermined time after which the dye is fixed by exposing the tissue sample to UV light. Imaging can then proceed at a later time since the dye molecule has been fixed into the tissue with little or no further diffusion possible. Depth of fixation can be controlled by the wavelength and intensity of the emitted light employed and the control of the molar absorptivity of the dye molecule itself in the UV region of excitation. Fixation of the dye can also be controlled by other means including such methods as heat activation by e.g. infrared illumination, microwave, and/or radiation.

Another example of depth fixation can result from changes in the diffusivity of a dye in the selected tissue. The diffusion coefficient of a compound generally depends on the combination of the molecular weight of the compound, the medium in which it diffuses, the temperature of the medium, and other variables known to those skilled in the art. Changes in the diffusive media can result in significant changes in the diffusion coefficient effectively fixing the dye in one locale for a period of time sufficient for the imaging methods herein described. In one example, the sample is immersed in a supercritical fluid such as carbon dioxide which contains the dyes of interest. The diffusion coefficient for such dyes in the supercritical fluid is much higher than that observed for the dyes in water. After a specified period of time the supercritical fluid conditions may be modified to, for example, bring the supercritical fluid to the liquid state. This may reduce the diffusion coefficient by orders of magnitude and effectively slow the diffusion of the dye throughout the tissue mass. This exemplary process allows for an image to be recorded where dye diffusion is no longer a determining variable in depth of penetration.

In another example of control of depth of penetration the dye molecule is derivatized to increase the molecular weight of the compound. The diffusion coefficient may be directly related to the molecular weight of the diffusing molecule. Increases in molecular weight may provide for a decrease in the diffusion coefficient for any molecule under comparable conditions. Increasing the molecular weight of the dye may therefore result in a significant reduction of the rate of penetration of the dye into the tissue sample. The molecular weight of a dye may be readily modified depending upon the dye and the available reactive groups available. Exemplary molecular additives to the dye molecule include the addition of substances such as polyethylene glycols of various molecular weights. Many other chemical substances can be covalently bonded to the dye molecule to increase its molecular weight and are apparent to those skilled in the art.

In yet another example of dye fixation with depth of penetration the tissue can be exposed to a dye in a liquid medium. After an appropriate amount of time has elapsed for the dye to penetrate to a depth of, for example, 5 microns, the temperature may be reduced thereby reducing the diffusion coefficient by orders of magnitude. Means by which the sample temperature can be cooled include treatments with such substances as liquid carbon dioxide, liquefied nitrogen and the like. Care should be exercised so as not to cause freeze rupturing of the cells in the sample to maintain structural integrity of the cellular mass. Useful solvents or co-solvents in such systems may include solvents or co-solvents that will not freeze until temperatures are below the freezing point of water such as glycols, alcohols and the like that are compatible with the tissue and dye.

In another implementation, superficial or surface staining may be used to achieve depth discrimination by controlling the depth to which the dye stain penetrates a sample under examination. Control of the depth of penetration of the dye reagent allows for the effective sectioning of the tissue by controlling the depth and the width of the penetrating dye. For example, a 5 micron section may be desired at a depth of 10 microns from the surface of the lesion under study. To control for depth of penetration and thickness of the dye band to achieve an effective 5 micron cross sections various methods may be employed. Some methods to achieve this result rely upon the controlled elimination of dye color with depth since tissue staining will occur at all depths as the dye penetrates the sample. Elimination of dye color or fluorescence may be accomplished through mechanisms such as photobleaching of the dye. In this exemplary process, high intensity light of selected wavelength is used to render the chromophoric moiety colorless or without fluorescence. Photobleaching may be accomplished through modification of the chemical structure of the dye, use of very high intensity light sources to cause the photodegradation of the dye, or through the use of a light activated photobleaching substance. In the latter case, photoreactive substances are allowed to diffuse into the sample. Subsequently, the sample may be exposed to a light source causing the photodegradation of the bleaching substances which reacts with the dye molecules. Exemplary substances include various peroxides and/or azides. Additional compounds that will form reactive species upon exposure to light of the proper wavelength are known to those skilled in the art. Such reactive moieties will react with the dye molecules with concurrent loss of their spectral properties.

In another implementation, two dyes may be administered concurrently too allow for both cytoplasmic and nuclear staining. In this implementation, the penetration process may be configured so that both dyes penetrate to a similar depth thus providing a single staining band for both dyes. As previously noted, the penetration process may be controlled by modulating the molecular weight of both dyes to achieve a similar penetration depth. Alternatively, or in addition, the depth of penetration may be controlled by modulating temperature, solvent, co-solvents, pH, osmotic state and the like such that both reagents diffuse into the sample in a similar manner.

Using superficial surface staining approaches, imaging speed is increased because a scanning laser would not be required. Instead, this approach controls the penetration depth of the stains so that fluorescent emissions in the desired wavelengths only occur at or near the surface of the sample. In some aspects, it may be desirable to separate excitation energy from emitted energy either in wavelength, time, or both. The resulting system may use LED illumination and a CMOS imager for detection with bandpass filters for the desired emission wavelengths allowing for the capture of a scattered light image. Surface staining can be accomplished by any method known to those skilled in the art. The following are provided as exemplary mechanisms for controlling or modifying the depth of penetration associated with a tissue sample when contacted with a reagent:

1. The diffusion time and soak conditions may be controlled so that reagent(s) penetrate to a pre-determined depth. Factors influencing penetration include tissue type, radius of curvature, local surface features such as cracks, pores, ducts, and follicles, as well as temperature.

2. A reagent may be linked to a molecule that has been chemically-modified to remain at or near a target surface contacted by the conjugate.

3. A reagent may be linked to an activator or quencher such that the reagent fluoresces when activated or de-quenched by a process that is depth-controlled.

4. The reagent may be contacted with a target surface by rolling or pressing the reagent onto the surface using a platen that has been conditioned to hold a carefully controlled amount of reagent. Like lithography, the stain's penetration depth may be controlled by limiting the amount of reagent available per unit surface area of the target surface.

5. An independent surface marking compound may be applied to a target surface using one of the approaches described above. This compound may be visible in a different wavelength of emission or illumination (or both) than the reagent. A system described herein may use this information to locate a depth discriminating element relative to the surface and thus capture emissions associated with a predetermined depth.

6. Multiple images may be captured at different time points in the penetration process. Later time points will generally represent deeper reagent penetration. Subsequent image analysis techniques may be used to deconvolve the surface fluorescence from deeper fluorescence.

Embodiments of the methods and systems described herein are applicable for the identification of objects of interest, such as neoplastic cells, associated with a tissue sample obtained from a subject having, or believed to have, a cell proliferation disorder. In one example, a tissue sample may be excised from the skin of a subject. In other examples, the tissue sample is obtained from breast, lymph, lung, or any other tissue suitable for microscopic examination by the implementation of a method or system described herein. A target surface may be examined microscopically to determine if the margins associated with the target surface are substantially clear margins of objects of interest. In one example, a tissue sample generated during Mohs' surgery (see Example below) generally comprises a two-dimensional (2D) specimen of about 0.5 cm² to about 2 cm². In another example, a breast lumpectomy tissue sample (see Example below) may be about 30 mm in major axis and may have surface area in excess of about 30 cm². In some implementations, tissue processing time (e.g., immobilizing the tissue sample in a carrier and contacting the sample with one or more reagents suitable for facilitating the imaging of the target surface) and image acquisition time (e.g., acquiring images the target surface and identifying an object of interest associated with the target surface) are functions of the area of the sample surface under examination. Sample processing and image acquisition time may be less than about 50, 40, 30, 20 or 10 seconds per cm² of imaged target surface. It is understood that the acquisition time may depend upon the resolution of the area under examination and the scanning rate of the detector assembly. In some implementations, the total time from sample immobilization to the initiation of image acquisition is less than about 50, 40, 30, 20, or 10 seconds per cm² of imaged target surface.

Implementations of the methods and systems described herein reduce the time from resection to imaging to about 3, 4, 5, 6, 7, 8, 10, 12 or 20 minutes and to be operable with minimal training through automation of sample handling, imaging, and image processing. In some implementations, the methods and systems provided herein eliminate the need for tissue sectioning, such as frozen tissue sectioning or paraffin-embedded tissue sectioning. In some implementations, methods and systems described herein utilize fluorescent confocal microscopy and special stains to image a target surface directly. The images may then be assembled into a pseudo-color image that simulates a traditional hematoxylin and eosin (H&E) stained section.

A health care professional may excise or resect a presumed lesion from a patient and examine the surgical margin for evidence of cancerous cells. In general, the procedure includes marking the surgical site with an identifier so that the sample may be oriented to the surgical site during scanning and examination. Accordingly, the term “identifier”, as used herein, is defined as any mark or fiducial associated with a surgical site that may be used to orient a tissue sample obtained from the surgical site. An identifier may be a synthetic identifier applied to the surgical site by a health care professional. Exemplary synthetic identifiers include, but are not limited to, ink or paint marks, whether visible or non-visible to the naked eye, applied to the surgical site. In other implementations, the identifier is a virtual identifier. Methods for implementing virtual identification of the orientation of a tissue sample include, but are not limited to, positioning the surgical site in a location in the procedure room that is electronically monitored by a positioning system that is local or global. In other implementations, the identifier is a natural identifier associated with the subject. Exemplary natural identifiers include, but are not limited to, moles, tattoos, freckles or any other native mark associated with the surgical site.

Additional exemplary sample orientation strategies include painting a pair of stripes or a stripe and another orienting mark, extending beyond the intended area of resection, to identify a direction relative to the patient. This process may be automated capturing a digital image of the surgical site and orienting the sample in a carrier so that its coronal (or some other prearranged) end is nearest a reference point associated with the carrier. Alternatively, the sample may be imaged while it is in the sample handler, capturing the paint marks for later alignment with the original image of the surgical site. In addition, the sample may be imaged while associated with the carrier. The orientation of the tissue sample may then be matched with the orientation of an image of the surgical site prior to further processing. In some implementations, this may involve: 1) rotating and aligning the image; 2) designating points of the sample image and the corresponding points of the patient; or 3) aligning the sample with identifier(s) included with a carrier.

A tissue sample may be immobilized on a carrier suitable for immobilizing and optionally processing a tissue sample. In one implementation, a carrier provides an examination surface against which a target surface associated with a tissue sample is applanated. The examination surface may be comprised of optical glass or plastic suitable for imaging the target surface. In other implementations, a carrier may include an element suitable for manipulating the tissue sample to expose different areas of the target surface. Accordingly, a tissue sample may be permanently or non-permanently immobilized on or in a carrier. In other implementations, a carrier may include a spindle-assembly for suspending the sample in or on the carrier. The assembly may be detachably associated with the carrier. A carrier may further include a liquid, gel or semi-solid medium that supports the tissue sample. A surface of the carrier may be temperature or water adhesive activated to capture the specimen throughout the process. In other implementations, the surface may include clamps, pins, spindles or any other mechanism suitable for facilitating tissue handling following excision. For example, a conventional three axis positioning stage coupled mechanically to the specimen tray may be used to move the specimen in front of the microscope and tile the imaged fields. In implementations where at least one surface of a sample will not be imaged, e.g., the epidermal surface of a skin sample, it is possible to applanate the tissue sample by compressing it between two surfaces, at least one of which is transparent and planar for imaging. Moving the surfaces relative to each other will roll, compress, or stretch the sample so that all portions of interest contact the imaging plane.

In other implementations related to dermatological samples, a sample may be affixed epidermis side down to a carrier surface as described herein. A detector assembly may the use surface following techniques to image the entire surface of interest without making it planar.

Methods and systems provided herein enable health care professionals to identify objects of interest associated with a tissue sample. Objects of interest include individual and groups of cells having, or believed to have, a cell proliferative disorder. Such cells are generally indicative of cancer, such as skin cancer or breast cancer. Typically a tissue sample will include proteins, polynucleotides, organic material, cells, and any combination of the foregoing. In general, a tissue is a mass of connected cells and/or extracellular matrix material (e.g., skin tissue, CNS tissue, neural tissue, eye tissue, placental tissue, mammary gland tissue, gastrointestinal tissue, musculoskeletal tissue, genitourinary tissue, and the like) derived from, for example, a human or other mammal and includes the connecting material and the liquid material in association with the cells and/or tissues.

As discussed below, tissue samples are analyzed by acquiring an image of a target surface using a system provided herein. In some implementations, the system includes a microscope suitable for examining a target surface at various magnifications. For example, when an image is acquired and candidate object of interest identified, such candidate objects can be further analyzed at a higher magnification. In some implementations, the microscope is a confocal microscope, such as a confocal laser fluorescence microscope. A system may further include a radiation source suitable for irradiating a target surface at various wavelengths, including between about 500 nm and 530, and between about 620 nm and 640 nm.

A method provided herein may include capturing images of one or more regions of the target surface and processing the images to produce a composite image. Once an image or composite image is obtained, the position of an object of interest relative to the surgical site may be obtained by correlating the position of the identifier associated with the sample and the position of the identifier associated with the surgical site.

Information generated in the performance of a method provided herein may be displayed on a display port and stored in a programmable processing device. In some implementations, the processing device is associated with the display port. In other implementations, the processing device stores information associated with a subject from which a tissue sample under examination was obtained. Subject information may include age, gender, blood pressure, heart rate, and any other current or prior medical or personal history relevant to subject treatment. This information may be correlated with the images generated by examination of a tissue sample derived from the subject.

In some implementations, the programmable processing device is a server configured to maintain a database of images and to provide access to the images to a remote user through a network.

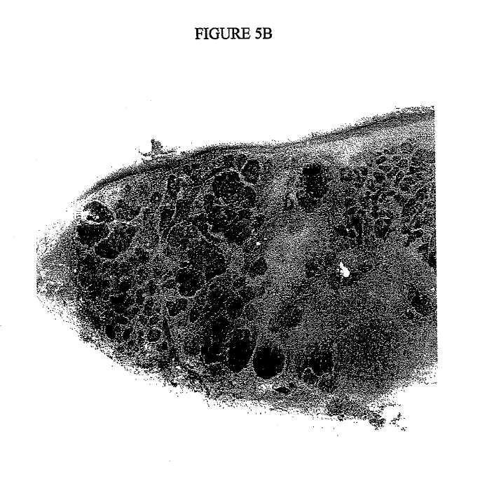

In another embodiment, images obtained from a method described above are transmitted to a remote user that verifies the presence of an object of interest in any of the images. The remote user further identifies the image, or plurality of images, that best depicts the position of the object in the tissue sample and correlates the identification with the identifier to determine the position of the object relative to the surgical site. The remote user optionally annotates the image or plurality of images identified by delineating the detected object of interest. An annotated image that includes at least one demarcation alerting a health care provider to the position of the detected object is transmitted to a health care provider. In one implementation, the remote user generates a report which is made available to a health care provider present at the location of the surgical procedure. The annotated images may be presented to the health care provider in a montage format or as individual images comprising identified objects of interest. The images may be further correlated with the subject information discussed above. In another implementation, the remote user and health care provider at the surgical procedure location may communicate in real time by viewing and annotating the same images/information simultaneously. Exemplary images generated by methods and systems provided herein are illustrated in FIG. 5A and FIG. 5B. Such images may be stored, processed, annotated, and transmitted by various mechanisms known to the skilled artisan and described in this application.

In another embodiment, a method for substantially reducing the amount of time needed to prepare and image an excised tissue sample is provided. The method includes preparing and imaging the sample by immobilizing the tissue sample in a carrier suitable for manipulating a target surface associated with a field of view and contacting the sample with one or more reagents suitable for facilitating the imaging of an object of interest by microscopy. In some aspects, the microscopy is fluorescence microscopy. In other aspects, the sample is not sectioned or substantially modified by a pliability-reducing treatment. Methods and systems provided herein, and exemplified in the examples set forth below, substantially reduce tissue processing and imaging time as currently practiced by health care professionals. While not bound by a specific means for imaging a sample processed by a method provided herein, in general such methods further include irradiating the target surface at one or more wavelengths selected to excite the one or more reagents associated with the target surface and acquiring images of one or more regions of the target surface. Various methods for identifying an object of interest associated with the target surface, displaying the object of interest on a display, and optionally storing the acquired images, are known to those skilled in the art and are applicable to the methods provided herein.

In another embodiment, a system for processing and imaging a tissue sample is provided. In general, a system includes a carrier suitable for manipulating a target surface associated with a tissue sample obtained from a surgical site and a radiation source configured to direct input radiation, at one or more wavelengths, to a target surface associated with the tissue sample. A detector assembly configured to capture radiation generated by the reagents associated with the target surface is also included. Optionally, the system includes a controller operably associated with the detector assembly. In some implementations the controller is configured to acquire a plurality of images of one or more regions of the target surface. A system also includes a display element associated with the detector assembly and controller. The display element allows a user to visualize an object of interest and correlate the position of the object with the identifier to determine the position of the object of interest relative to a surgical site. In other implementations, a system optionally includes a storage element for storing imaging information obtained from the detector assembly.

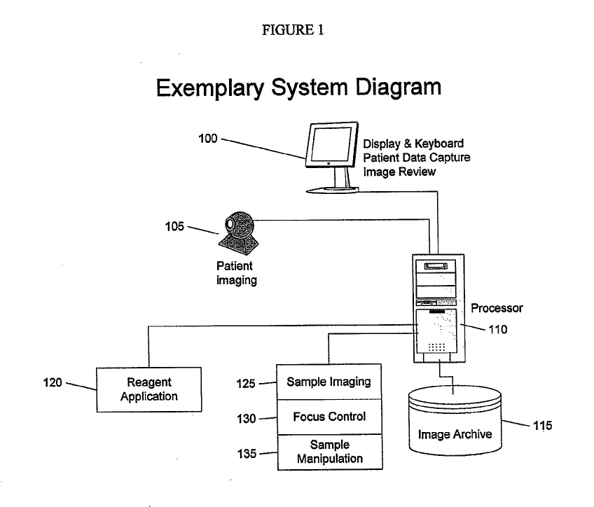

Referring to FIG. 1, a surgical site associated with a subject, such as a patient undergoing a surgical procedure, may be imaged by a patient imager 105. Patient imager 105 may be any device suitable for capturing an image of a surgical site, such as an analog or digital camera. Patient imager 105 may be physically or wirelessly connected with processor 110 so that images of a surgical site may be correlated with other patient data stored by processor 110. A system can comprise a controller having at least one processor 110. The system may further include a computer/image monitor 100 and other external peripherals including a storage device for archiving images 115, a pointing device, such as a track ball or mouse device, a user input device, such as a touch screen, keyboard, or voice recognition unit and color printer.

A system further includes a detector assembly for sample imaging 125. A detector assembly may include a radiation source for irradiating a target surface associated with a tissue sample at one or more wavelengths. The radiation source may be a laser, such as a gas laser, a solid-state laser, a tunable dye laser, or semiconductor laser. A radiation source may emit radiation at virtually any wavelength needed to visualize an object of interest associated with a tissue sample. In some implementations, the wavelength may be in a range of about 200 nm to 2000 nm, or in the range of about 390 nm to about 750 nm. It is understood that the radiation source may be a fluorescent excitation source and may further include a plurality of fluorescent filters on a turret or wheel. Alternatively, a filter wheel may have an electronically tunable filter.

A detector assembly may further include a microscope and a stage suitable for securing a carrier comprising a tissue sample in a field of view. Microscope movements may be under the control of processor 110 through a number of microscope-subsystem functions. In some implementations the microscope is a confocal microscope. In other implementations a microscope may be a laser confocal fluorescent microscope. For example, two fluorescent dyes and a two-color laser scanning confocal microscopes may be used. Software may be used to correlate the images from each excitation wavelength in false color to create a simulation of an H&E stained section. The optical chain can focus from the sample surface to up to 50 microns deep in order to support depth scanning for analysis of ducts from, e.g., a mammary gland biopsy.

Table 1 provides a list of exemplary microscope specifications:

Microscope specs

| | |
|---|---|
| Kind of imager | Point scanning laser confocal microscope |
| Imaging sensor | PMT |
| Wavelengths | 514 nM (Eosin), 633 nM (DRAQ) |
| Scanning rate | 0.2-0.5 cm²/sec (per wavelength) 200-400 Hz |
| Optical field | 1.5 (10x)-3 mm (5x), as needed to meet |
| of view | other specs |
| Field flatness | better than 10 uM |
| over FOV | |
| Dynamic range | 12 bit or better ADC per channel, strictly |
| | monotonic |
| Numerical aperture | 0.3 or better desired, air coupled - driven by |
| | imaging sensitivity and depth scanning. Plan |
| | Flour 10X air NA 0.3 is an exemplary lens. NA |
| | 0.3 will give a 5 um optical slice. |
| Spot size | ~2 uM |
| Effective Pixel size | ~2 uM |
| Image format | DICOM compliant |
| Depth resolution | 5 uM virtual section |
| Depth settings | Surface and 50 uM, program selectable |
| Scanning stage for | XY scanning stage (20 mm min. travel each |
| tiling | direction) |
| Z axis drive for | Standard focus drive for confocal scanning or |
| optical sectioning | piezo-driven objective for faster Z scanning. |
| of tissue | Travel: 75 uM min. |
| Focusing | Autofocus servo by pre-scanning carrier surface |
| | Non-penetrating third dye |
| | Incident light contrast autofocus on a per-tile basis |
| | Use focus strategy to remove need to applanate the |
| | sample |

In another implementation, a detector assembly may include a Total Internal Reflection Fluorescent Microscope (TIRFM). In general, TIRF uses grazing illumination of an optical flat surface in contact with the sample to create an evanescent wave that penetrates the sample to a depth of about 100 nM. In contrast to a confocal microscope, TIRF achieves depth discrimination by illuminating a thin section of the surface of the sample.

It is understood that processor 110 and/or image archive 115 may store images obtained from both patient imager 105 and sample imaging array 125. The controller may be configured to determine a surface profile of a target surface based on a signal from the detector assembly. Referring again to FIG. 1, focus controller 130 determines a surface contour associated with a target surface and continuously modifies the detector assembly so that the surface is in focus. For example, focus controller 130 may modify the focus of a microscope or other imaging device associated with detector assembly. Such other imaging devices include a complementary metal oxide semiconductor (CMOS) imager, a charge coupled device (CCD) imager, a camera with photosensitive film, a Vidicon camera, or any combination thereof. In some implementations, the controller is optionally programmed to automatically identify an object of interest associated with the target surface. Accordingly, a system provided herein may determine the surface contour of a tissue sample and dynamically position the sample and scanning objective relative to each other such that all image pixels or image tiles are in a consistent depth relationship to each other and to the design center of the optical system.

A typical microscopic imaging system with constrained depth of field or depth discriminating optics relies on the assumption that the sample under view is planar to a tolerance that is much less than the depth of field or depth discrimination window of the optical system. Focusing may be accomplished by mechanical alignment, or a movable Z stage with one of several known focusing strategies, or by taking multiple slices at various Z positions and selecting those parts of the resulting image that are in focus according to some (possibly independently) measured or computed parameter.

In one implementation, a surface contour may be determined by using structured light. This approach involves the use of one or more beams of light that are projected in a known relationship to an imaging system such as a microscope. By imaging the location and shape of the beam as it contacts the sample, the system can calculate the distance from those portions of the sample surface to the objective using prior knowledge. In another implementation, a surface contour may be determined by projecting a collimated beam at an angle to the axis of the imaging system so that it projects a spot on the sample whose lateral position corresponds to the distance from the sample surface to the objective. In another implementation, a surface contour may be determined by projecting a hollow cone of light with a large solid angle, where the cone necks at the focal point of the imaging system. By moving the sample until the projection of the cone on the sample becomes a point, the system may effectively focus that point.

As noted above, a detector assembly may include various devices for imaging a tissue sample. Examples of such devices include, but are not limited to, microscopes, confocal microscopes, fluorescent confocal microscopes, and or other devices suitable for conducting colorimetric methods, spectroscopic methods, resonance based methods, or substantially any combination thereof. In some embodiments, a detector assembly may be located in the area where a surgical procedure is conducted. Alternatively, the detector assembly may be located in a remote location. A detector assembly may include a high quantum efficiency digital camera to capture transmitted and fluorescent images. In some implementations, a Hamamatsu Orca-AG deep-cooled 1,344×1,024 pixel 12-bit digital CCD camera with digital (fire wire) output can be used.

Numerous types of users, e.g., health-care providers, may interact with a system provided herein. In some embodiments, a user may be human. In some embodiments, a user may be non-human. In some embodiments, a user may interact with one or more detector assemblies, one or more display units 100, one or more user interfaces, one or more processors 110, and/or substantially any combination thereof. The user can interact through use of numerous types of user interfaces such as keyboards. For example, one or more users may interact through use of numerous interfaces that utilize hardwired methods, such as through use of a keyboard, use of wireless methods, use of the internet, and the like. In some embodiments, a user may be a health-care provider. Examples of such health-care providers include, but are not limited to, physicians, nurses, pathologists, and the like.

A system provided herein may include one or more display units 100. Numerous types of display units 100 may be used in association with the system. Examples of such display units 100 include, but are not limited to, liquid crystal displays, printers, audible displays, cathode ray displays, plasma display panels, and the like.

In some embodiments, one or more display units 100 may be physically coupled to one or more detector assemblies. In some embodiments, one or more display units 100 may be remotely coupled to one or more detector assemblies. For example, in some embodiments, one or more display units 100 may receive one or more signals from one or more detector assemblies collecting data (e.g., images) related to a tissue sample, or multiple tissue samples. Accordingly, one or more display units 100 may be positioned in one or more locations that are remote from the position where analysis of one or more tissue samples takes place. Examples of such remote locations include, but are not limited to, the offices of physicians, surgeons, pathologists, nurses, technicians, and the like.

The system may include one or more processors that include recording units for storing images and other data. In some embodiments, one or more recording units can communicate with one or more detector assemblies, one or more display units 100, one or more user interfaces, and/or substantially any combination thereof. Many types of recording units may be used within a system. Examples of such recording devices include those that utilize a recordable medium that includes, but is not limited to, many types of memory, optical disks, magnetic disks, magnetic tape, and the like.

Referring again to FIG. 1, in some embodiments, processor 110 may be connected (physically or wirelessly) with a device for reagent application 120. One or more devices for reagent application 120 may be employed to control or automate the process of contacting a tissue sample with a reagent. In other embodiments, processor 110 may be connected (physically or wirelessly) with a device for sample manipulation 135. It is understood that the sample may or may not be associated with a carrier during the reagent application process.

The methods and systems provided herein reduce the amount of time needed to process and image a sample. The reduction in time may be due, in part, to a novel process for imaging the tissue without limiting the pliability of the sample. Accordingly, in some implementations, methods provided herein do not involve a “pliability-reducing treatment” step during the processing of a sample. As used herein, a “pliability-reducing treatment” means any chemical- or temperature-related treatment that results in a tissue sample that is less pliable (i.e., more rigid) than its native state. Exemplary pliability-reducing treatments include fixing the tissue sample in wax or paraffin and/or freezing the tissue sample.

A system provided herein may further include software that analyzes areas of interest in order to provide a technician or user additional information, such as types of objects and morphological factors, the state of objects, and/or other important visible information that would help the user make an informed decision regarding a sample. For example, a technician or health care provider may want to analyze a sample to determine the presence cancer cells. The user may also want to know in what quantity the cells are present and whether they are in a state of apoptosis.

In some implementations, images generated by a method system provided herein may be deconvolved. In general, deconvolution is a process for removing out-of-focus photons from an image to give a higher resolution, e.g., a “restored” image. Deconvolution is a post-acquisition image processing technique which can use simple algorithms that “deblur” a 2-D image or algorithms that “restore” a 3-D image stack to a high resolution image. It is understood that deconvolution can be utilized to further process both confocal images and wide-field images. For example, the skilled artisan will recognize that any image acquired on a digital fluorescence microscope can be deconvolved. In addition, deconvolution techniques may be used to process transmitted light images collected under a variety of contrast enhancing strategies. Deconvolution may also be used to modify three-dimensional montages constructed from a series of optical sections.

The most commonly utilized algorithms for deconvolution in optical microscopy can be divided into two classes: deblurring and image restoration. Deblurring algorithms are fundamentally two-dimensional, because they apply an operation plane-by-plane to each two-dimensional plane of a three-dimensional image stack. In contrast, image restoration algorithms are properly termed “three-dimensional” because they operate simultaneously on every pixel in a three-dimensional image stack.

Resolution in optical microscopy is often assessed by means of an optical unit termed the Rayleigh criterion, which was originally formulated for determining the resolution of two-dimensional telescope images, but has since spread into many other arenas in optics. The Rayleigh criterion is defined in terms of the minimum resolvable distance between two point sources of light generated from a specimen and is not dependent upon the magnification used to produce the image. In deconvolution analysis, the three-dimensional nature of the point spread function must be considered when applying the Rayleigh criterion.

It will be appreciated that electronic and software applications may involve dynamic and flexible processes and thus that illustrated blocks can be performed in other sequences different than the one shown and/or blocks may be combined or separated into multiple components. In some examples, blocks may be performed concurrently, substantially in parallel, and/or at substantially different points in time. Further aspects of the methods and systems described herein are illustrated in the Examples discussed below.

### EXAMPLES

### General System Application Workflow:

1. Patient imaging—capture an image of the surgical site and the identifiers prior to resection. The system correlates the image of the pre-excision surgical site with sample images generated during step 5 below so as to guide the doctor if more tissue needs to be removed. A CCD camera captures the image and the system stores it for later use.

2. Patient data capture—setup screen captures at minimum the patient ID, the pre-operative site image for later orientation, timestamp, and optional notes.

3. Sample Handling, Orientation, & Applanation—place resected tissue in a sample carrier. The carrier compresses the tissue so that the margin area is applanated against optical plastic or glass. The carrier inserts into the reader for imaging, and can be manipulated so that the tissue sample rolls to expose more of the margin if necessary. Tissue is oriented by the identifiers applied to the epidermis.

4. Sample Staining—contact tissue sample with reagents such as Eosin and DRAQ5. Reagents are chosen to stain the same cellular components as stained by light microscopy histology stains (hematoxylin and eosin) currently used in Mohs surgery and to emit at distinct wavelengths. One may preferentially stain DNA (DRAQ5), and the other (Eosin) preferentially stains cytoplasm. Reagent chemistry may be designed to minimize time and number of steps required to prepare the sample for imaging.

5. Sample Imaging—Confocal fluorescent scanning microscope with a low noise, high sensitivity PMT detector. Additional characteristics may include:
Two laser wavelengths (633 & 514 nM—prefer solid state or HeNe)
Detector: low noise PMT; 12-16 bits per pixel A/D conversion
Single beam point scanning
Image step size: 3 mm² with 5×, 1.5 mm² with 10×
Field of view: 3 mm² with 5×, 1.5 mm² with 10×
Spot size: ˜1 micron (diffraction limited) at tissue surface
Resolution: pixel dimensions=3×3 microns in 1024×1024 image.

In general, the resolution of a confocal microscope may be calculated as follows: Rₗₐₜₑᵣₐₗ=0.6/NA or 2 microns at 500 nm with 0.15 NA, and 1 micron at 500 nm with 0.3 NA; or Rₐₓᵢₐₗ=approximately 20 microns with 0.15 NA air lens at 500 nm; approximately 5 microns with 0.3 NA.

6. Image presentation and analysis support—display image in pseudo-color simulating H&E stained thin section on a flat panel monitor. The image is aligned relative to the pre-operative patient image to make it easy for the surgeon to determine where to take additional margin.

### Mohs' Surgery Application Workflow:

1. Make identifier marks on surgical site, extending onto the area to be resected. Capture an image of the site for later orientation to sample.

2. Excise the lesion, and make a relaxing incision. Insert sample into staining carrier.

3. Insert carrier into stain station.

4. System stains with exemplary reagents Eosin and DRAQ5.

5. Insert sample into imaging carrier with epidermis facing away from the optical flat. Adhere sample to the optical surface and remove air bubbles.

6. Insert carrier into imaging station.

7. System images the sample and converts to an H&E equivalent image for presentation to histopathologist.

8. View result image, annotate, and decide whether to remove more tissue—system shows image in patient context using identifier marks to determine where to remove more tissue if necessary.

9. System designates on the patient the area for further removal, if necessary.

10. Archive the image, and optionally archive or dispose of the tissue sample.

### Lumpectomy Tissue Processing:

In the case of lumpectomy, a surgeon excises a suspicious lump from, for example, breast tissue. A pathologist examines suspicious regions of the excised lump for cancerous tissue on the margin. The pathology exam generally focuses on ductal tissue, where cancer tends to occur, as opposed to adipose tissue, which makes up the majority of breast tissue. The patient waits days to weeks for a result, and there is a possibility of re-operation. The recurrence rate of cancer under the current standard of care is about 35%, so there is considerable opportunity to improve outcomes. Problems with the current approach include: 1) the pathologist does not examine the entire surface of the lump, so it is possible to miss cancerous tissue; and 2) the patient waits for results after the procedure, causing unnecessary stress.

Systems provided herein would improve diagnosis and treatment by scanning the entire surface of the lump at sufficient resolution to identify all areas with concentrations of nuclei by marking the locations of concentrations of nuclei (which fluoresce at a different wavelength than surrounding tissue because of the nuclear stain). Standard blob analysis algorithms, tuned to look for circular or elliptical blobs on a two-color fluorescence scan or a scattered light micrograph, can also identify ducts. Areas of unusual nuclear concentration or morphology could also be identified by blob analysis or by statistical examination of fluorescence data corresponding to the nuclear affinity dye. A system capable of imaging tissue obtained from a lumpectomy may image the entire surface of an irregular spheroid lump of soft, elastic tissue. Exemplary mechanisms include:

1. Impale the tissue on a spindle that has a single needle, two or more parallel needles, or one or more needles that optionally include anti-rotation barbs, such that the tissue is positively oriented with respect to the axis of rotation of the spindle. The needles may be of varying lengths, and may have features intended to support the tissue from sagging as it is rotated. The spindle may be vertical, horizontal, or neither, and may also have additional degrees of freedom so that the entire tissue surface is visible with minimal occlusion from the spindle itself. The spindle may be longer than the tissue sample, and supported by a manipulator that can revolve or translate (or both) to facilitate imaging.

2. Applanate the tissue between two planes, at least one of which is optically transparent. By moving the other sheet while maintaining pressure on the lump, the lump is forced to roll, exposing its entire surface for examination.

3. Immerse the tissue in a cuvette containing an optically transparent fluid. The fluid's refractive index may match that of the cuvette, and its density (mass/volume) may match that of the sample. The cuvette may include at least one flat surface through which the imaging system can image the sample. The spindled tissue is immersed in the cuvette from the open top—this obviates the need for a fluid-tight seal or magnetic coupling in the bottom of the cuvette—though either of these would also work. The fluid bath increases the effective numerical perture (NA) of the optical system. Its density may be chosen to approximate that of the tissue which also reduces the tendency of the tissue to sag on the spindle thereby reducing motion related artifacts.

Tissue from Lumpectomy Application Workflow:

1. Make fiducial marks on the surgical site (see open issues). Capture an image of the site for later orientation to sample.

2. Excise the lump, impale it on a disposable handling spindle.

3. Insert the spindle into the stain station.

4. System performs a superficial stain with Eosin and DRAQ5.

5. Insert sample into the imaging station.

6. Insert carrier into imaging station.

7. System images the sample in white light to capture the identifiers.

8. System captures a two-color fluorescent image using one surface tracking techniques known to the skilled artisan to maintain focus and brightness.

9. System identifies regions of the image corresponding to transected ducts automatically, and stores the image-relative positions of these regions.

10. System converts to an H&E equivalent image for presentation to histopathologist.

11. Display annotated image to the operator.

12. Optionally transport image across a network and present for remote histopathology. Remote interface allows the histopathologist to designate some or all areas of the image for detail scanning.

13. Optionally re-scan designated regions of interest at higher resolution or alternate depths.

14. View result image, annotate, and decide whether to remove more tissue—system shows image in patient context using fiducial marks so that it is easy to decide where to remove more tissue if necessary.

15. System designates on the patient the area for further removal, if necessary.

16. Archive the image, and optionally archive or dispose of the tissue sample.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method comprising:
a) capturing an image of a surgical site associated with a subject, wherein the surgical site comprises at least one identifier suitable for orienting a tissue sample associated with the surgical site;
b) excising the tissue sample;
c) immobilizing the tissue sample in a carrier suitable for manipulating a target surface associated with a field of view;
d) prior to, concomitant with, or subsequent to part c), contacting the sample with one or more reagents suitable for facilitating the imaging of an object of interest by fluorescence microscopy;
e) irradiating the target surface at one or more wavelengths selected to excite the one or more reagents associated with the target surface;
f) acquiring images of one or more regions of the target surface and identifying an object of interest associated with the target surface;
g) using the identifier to determine the position of the object of interest relative to the surgical site;
h) displaying the object of interest on a display; and
i) optionally storing the information off) and g).

2. The method of claim 1, wherein the identifier is a synthetic identifier.

3. The method of claim 1, wherein the identifier is a virtual identifier.

4. The method of claim 1, wherein the identifier is a natural identifier associated with the subject.

5. The method of claim 1, wherein the tissue sample is suspected of comprising cells having a cell proliferative disorder.

6. The method of claim 3, wherein the cell proliferative disorder is a neoplasm.

7. The method of claim 4, wherein the cell proliferative disorder is skin cancer or breast cancer.

8. The method of claim 1, wherein the immobilizing is by suspending the sample on a spindle-assembly associated with the carrier, wherein the target surface is positioned for imaging.

9. The method of claim 8, wherein the spindle-assembly is detachably associated with the carrier.

10. The method of claim 8, wherein the carrier comprises a medium that supports the tissue sample.

11. The method of claim 10, wherein the medium comprises a liquid, gel or semi-solid.

12. The method of claim 1, wherein the immobilizing is by contacting the sample with an applanation surface associated with the carrier and suitable for imaging the target surface in contact with the applanation surface.

13. The method of claim 12, wherein the applanation surface is comprised of optical plastic or glass.

14. The method of claim 1, wherein the one or more reagents are fluorescent dyes.

15. The method of claim 14, wherein the fluorescent dyes are selected from the group consisting of Eosin, DRAQ5, SYTO® and LDS 751®.

16. The method of claim 1, wherein the object of interest is a cell.

17. The method of claim 1, wherein the object of interest is a nucleus of a cell.

18. The method of claim 1, where the fluorescence microscopy is laser confocal fluorescence microscopy.

19. The method of claim 1, wherein the fluorescence microscopy includes deconvolving a set of images taken with substantially narrow depth of field at different depths.

20. The method of claim 1, wherein the irradiating is at a wavelength between 500 nm and 700 nm.

21. The method of claim 1, wherein the sample is contacted with Eosin and DRAQ5.

22. The method of claim 21, wherein the sample is irradiated simultaneously or successively at between about 500 nm and 530, and between about 620 nm and 640 nm.

23. The method of claim 1, wherein the images of one or more regions of the target surface are processed to produce a composite image.

24. The method of claim 1, wherein identifying the object of interest associated with the target surface is automated.

25. The method of claim 1, wherein determining the position of the object of interest relative to the surgical site comprises correlating the position of the identifier associated with the sample and the position of the identifier associated with the surgical site.

26. The method of claim 1, wherein the information is stored in a programmable processing device associated with a display port.

27. The method of claim 26, wherein the programmable processing device is a server that is configured to maintain a database of images and to provide access to the images to a remote user.

28. The method of claim 27, wherein the remote user accesses the images through a network.

29. The method of claim 1, wherein the tissue sample is sectioned prior to part c).

30. The method of claim 1, wherein the sample is immobilized following part b).

31. The method of claim 1, wherein the total time from c) to f) is less than about 50 seconds per cm² of imaged target surface.

32. The method of claim 1, wherein the total time from c) to f) is less than about 40 seconds per cm² of imaged target surface.

33. The method of claim 1, wherein the total time from c) to f) is less than about 30 seconds per cm² of imaged target surface.

34. The method of claim 1, wherein the total time from c) to f) is less than about 20 seconds per cm² of imaged target surface.

35. The method of claim 1, wherein the total time from c) to f) is less than about 10 seconds per cm² of imaged target surface.

36. A method comprising:
a) transmitting the images obtained in claim 1, part f) to a remote user;
b) verifying the presence of an object of interest in any of the images;
c) identifying the image, or plurality of images, that best depicts the position of the object in the tissue sample and correlating the identification with the identifier to determine the position of the object relative to the surgical site;
d) annotating the image or plurality of images identified in c) by delineating the detected object of interest;
e) generating an annotated image highlighting the detected object of interest; and
f) transmitting the annotated image of e) to a health care provider, wherein the annotated image includes at least one demarcation alerting the health care provider to the position of the detected object.

37. A method comprising:
a) capturing an image of a surgical site associated with a subject, wherein the surgical site comprises at least one identifier suitable for orienting a tissue sample associated with the surgical site;
b) excising the tissue sample;
c) immobilizing the tissue sample in a carrier suitable for manipulating a target surface associated with a field of view;
d) prior to, concomitant with, or subsequent to part c), contacting the sample with one or more reagents suitable for facilitating the imaging of an object of interest by fluorescence microscopy, wherein the tissue sample is not frozen;
e) irradiating the target surface at one or more wavelengths selected to excite the one or more reagents associated with the target surface;
f) acquiring images of one or more regions of the target surface and identifying an object of interest associated with the target surface;
g) using the identifier to determine the position of the object of interest relative to the surgical site;
h) displaying the object of interest on a display; and
i) optionally storing the information off) and g).

38. A method comprising:
a) capturing an image of a surgical site associated with a subject, wherein the surgical site comprises at least one identifier suitable for orienting a tissue sample associated with the surgical site;
b) excising the tissue sample;
c) immobilizing the tissue sample in a carrier suitable for manipulating a target surface associated with a field of view;
d) prior to, concomitant with, or subsequent to part c), contacting the sample with one or more reagents suitable for facilitating the imaging of an object of interest by microscopy, wherein the one or more reagents penetrate the target surface of the sample to a predetermined depth;
e) irradiating the target surface at one or more wavelengths selected to excite the one or more reagents associated with the target surface;
f) acquiring images of one or more regions of the target surface and identifying an object of interest associated with the target surface;
g) using the identifier to determine the position of the object of interest relative to the surgical site;
h) displaying the object of interest on a display; and
i) optionally storing the information off) and g).

39. The method of claim 33, wherein the microscopy includes deconvolving a set of images taken with substantially narrow depth of field at different depths.

40. A method comprising:
a) capturing an image of a surgical site associated with a subject, wherein the surgical site comprises at least one identifier suitable for orienting a tissue sample associated with the surgical site;
b) excising the tissue sample;
c) immobilizing the tissue sample in a carrier suitable for manipulating a target surface associated with a field of view;
d) prior to, concomitant with, or subsequent to part c), contacting the sample with one or more reagents suitable for facilitating the imaging of an object of interest by microscopy, wherein the sample is not sectioned prior to contacting with the one or more reagents;
e) irradiating the target surface at one or more wavelengths selected to excite the one or more reagents associated with the target surface;
f) acquiring images of one or more regions of the target surface and identifying an object of interest associated with the target surface;
g) using the identifier to determine the position of the object of interest relative to the surgical site;
h) displaying the object of interest on a display; and
i) optionally storing the information off) and g).

41. A method comprising:
a) providing a surgical site associated with a subject, wherein the surgical site comprises at least one identifier suitable for orienting a tissue sample associated with the surgical site;
b) excising the tissue sample;
c) immobilizing the tissue sample in a carrier suitable for manipulating a target surface associated with a field of view;
d) prior to, concomitant with, or subsequent to part c), contacting the sample with one or more reagents suitable for facilitating the imaging of an object of interest by fluorescence microscopy;
e) irradiating the target surface at one or more wavelengths selected to excite the one or more reagents associated with the target surface;
f) acquiring images of one or more regions of the target surface and identifying an object of interest associated with the target surface;
g) using the identifier to determine the position of the object of interest relative to the surgical site;
h) displaying the object of interest on a display; and
i) optionally storing the information off) and g).

42. The method of claim 36, further comprising capturing an image of the surgical site prior to excision of the tissue sample.

43. A method comprising;
a) substantially reducing the amount of time required to prepare and image an excised tissue sample, the preparation and imaging including:
i) immobilizing the tissue sample in a carrier suitable for manipulating a target surface associated with a field of view;
ii) contacting the sample with one or more reagents suitable for facilitating the imaging of an object of interest by fluorescence microscopy, wherein the sample is not sectioned or substantially modified by a pliability-reducing treatment;
iii) irradiating the target surface at one or more wavelengths selected to excite the one or more reagents associated with the target surface and acquiring images of one or more regions of the target surface; and
b) imaging the object of interest.

44. The method of claim 43, wherein the total time from a) i) to a) iii) is less than about 60 seconds per cm² of imaged target surface.

45. The method of claim 43, wherein the total time from a) i) to a) iii) is less than about 50 seconds per cm² of imaged target surface.

46. The method of claim 43, wherein the total time from a) i) to a) iii) is less than about 40 seconds per cm² of imaged target surface.

47. The method of claim 43, wherein the total time from a) i) to a) iii) is less than about 30 seconds per cm² of imaged target surface.

48. The method of claim 43, wherein the total time from a) i) to a) iii) is less than about 20 seconds per cm² of imaged target surface.

49. The method of claim 43, wherein the total time from a) i) to a) iii) is less than about 10 seconds per cm² of imaged target surface.

50. The method of claim 43, wherein the total time to complete part a) i) and part a) ii) is less than about 10 minutes.

51. The method of claim 43, wherein the total time to complete part a) i) and part a) ii) is less than about 5 minutes.

52. The method of claim 43, wherein the total time to complete part a) i) and part a) ii) is less than about 2 minutes.

53. A system comprising:
a) a carrier suitable for manipulating a target surface associated with a tissue sample obtained from a surgical site, wherein the surgical site comprises an identifier suitable for orienting the tissue sample with the surgical site;
b) at least one radiation source configured to direct input radiation, at one or more wavelengths, to a target surface associated with the tissue sample, wherein the target surface comprises one or more reagents detectable by the input radiation;
c) a detector assembly configured to capture radiation generated by the reagents associated with the target surface;
d) a controller operably associated with the detector assembly, wherein controller is configured to acquire a plurality of images of one or more regions of the target surface;
e) a display associated with the detector assembly and controller, wherein a user visualizes the object of interest and correlates the position of the object with the identifier to determine the position of the object of interest relative to the surgical site; and
f) optionally storing the information of c) through e).

54. The system of claim 53, wherein the radiation source is a laser.

55. The system of claim 53, wherein the detector assembly comprises a laser confocal fluorescent microscope.

56. The system of claim 53, wherein the controller is configured to determine a surface contour of the target surface based on a signal from the detector.

57. The system of claim 53, wherein the controller is optionally programmed to automatically identify an object of interest associated with the target surface.

58. The system of claim 53, wherein the radiation source is a laser.

59. The system of claim 53, wherein the laser is a gas laser, a solid-state laser, a tunable dye laser, or semiconductor laser.

60. The system of claim 53, wherein the detector assembly comprises a complementary metal oxide semiconductor (CMOS) imager, a charge coupled device (CCD) imager, a camera with photosensitive film, a Vidicon camera, or any combination thereof.

61. The system claim 53, wherein the wavelength is in a range of 200 nm and 2000 nm.

62. The system of claim 53, wherein the wavelength is in a range of 390 nm and 750 nm.

63. The system of claim 53, wherein the detector assembly is operable to detect radiation both prior to and following application of a reagent.

64. A system comprising:
a) a means for capturing an image of a surgical site associated with a subject, wherein the surgical site comprises at least one identifier suitable for orienting a tissue sample associated with the surgical site;
b) a means for excising the tissue sample;
c) a means for immobilizing the tissue sample in a carrier suitable for manipulating a target surface associated with a field of view;
d) prior to, concomitant with, or subsequent to part c), contacting the sample with one or more reagents suitable for facilitating the imaging of an object of interest by fluorescence microscopy;
e) a means for irradiating the target surface at one or more wavelengths selected to excite the one or more reagents associated with the target surface;
f) a means for acquiring images of one or more regions of the target surface and identifying an object of interest associated with the target surface;
g) using the identifier to determine the position of the object of interest relative to the surgical site;
h) a means for displaying the object of interest on a display; and
i) a means for optionally storing the information off) and g).
